(19) United States
(12) Patent Application Publication
(10) Pub. No.: US 2010/0240051 A1
(43) Pub. Date: Sep. 23, 2010

(54) Device and Method for Preparing and Performing Multiple Polymerase Chain Reactions

(75) Inventors: Jeff Tza-Huei Wang, Timonium, MD (US); Yi Zhang, Baltimore, MD (US)

Correspondence Address: VENABLE LLP, P.O. BOX 34385, WASHINGTON, DC, 20043-9998, US

(73) Assignee: The Johns Hopkins University, Baltimore, MD (US)

(21) Appl. No.: 12/716,031

(22) Filed: Mar. 2, 2010

Related U.S. Application Data
(60) Provisional application No. 61/156,619, filed on Mar. 2, 2009.

(51) Int. Cl. C12Q 1/68; C12M 1/34; B05D 3/00

(52) U.S. Cl. 435/6; 435/287.1; 427/372.2

(57) ABSTRACT

The present invention relates to methods, kits and devices with multiple open reaction chambers having multiple pre-deposited primer compositions, and a basic sample loading mechanism that utilizes an immiscible companion fluid for preparing and performing multiple Polymerase Chain Reactions

This application claims priority to U.S. provisional application No. 61/156,619, filed Mar. 2, 2009, which is hereby incorporated herein by reference in its entirety.

The research resulting in the invention described herein was supported in part by Grant Numbers 1R21CA120742-01 and SPORE P50-CA058184-10 awarded by the National Institute of Health, Grant Numbers 0546012, 0730503 and 0725528 awarded by the National Science Foundation, and MF3 Micro/Nano Fluidics Fundamentals Focus (MF3) Center. The U.S. Government has certain rights in the invention.

### BACKGROUND

Although various methods are available for analyzing nucleic acids, polymerase chain reaction (PCR) remains one of the most widely used techniques. Many molecular biology protocols today still heavily rely on PCR or modified PCR. Currently, one of the major trends in biological and chemical analysis has been the development of microfabricated miniaturized platforms. In particular, great effort has been put into the miniaturization of genetic tests as they provide fast analysis, reduced reagent and sample consumption, and high portability. The most straightforward approach used involves fabricating an array of reaction chambers of micro, nano or even picoliter volumes for parallel PCR reactions. Alternatively, miniaturization is realized by performing PCR in free micro droplets on a modified surface. The droplets function as the virtual reaction chambers by providing fluidic confinement.

However, many current array based devices are designed for performing multiple PCR reactions in identical conditions with the same primers and DNA templates, thus limiting their use to singleplex analysis. Multiple gene screening with different primer sets still relies on separate sample loading via complex fluidic networks.

Furthermore, most designs concentrate on engineering perspectives, focusing on the miniaturization and system integration, while often overlooking practicality issues. As a result, very complicated microfluidic control modules are usually incorporated in order to cope with pressurization and micro bubble formation and expansion during thermal cycling. Unfortunately, such delicate devices often use too much or waste previous biological samples and require operation by experienced microfluidic personnel and thus are difficult to fit into routine biological laboratories and clinical settings. Hence they are not commonly adopted for use in PCR based diagnostic assays such as methylation specific PCR (MSP).

There is a need for an efficient, yet simple device that can easily be handled by individuals who do not have microfluidic experience.

The present invention provides a device with multiple open reaction chambers having multiple pre-deposited primer compositions, and a basic sample loading mechanism that utilizes an immiscible companion fluid that obviates these challenges.

This device is simple to handle and facilitates easy surface modification, primer deposition and sample retrieval while eliminating bubble formation and expansion. The device allows for multiple reactions and analysis with just single sample injection.

### SUMMARY OF INVENTION

The present invention relates to a device that may comprise a sampling chamber having an access point and connected to at least one or more channels extending radially outward, each of said channels terminating at an open reaction chamber containing a pre-deposited reaction material. In a particular embodiment the sampling chamber may be connected to about six channels. In another embodiment, each of the channels may branch off into two or more secondary channels, which may be connected to an open reaction chamber each. In another aspect, the device may have about 12 open reaction chambers.

In one aspect of the invention, the reaction material may be a pre-deposited solidified material, which may be a PCR primer, such as MSP primers. In another aspect, the primer may be specific for a bisulfite modified unmethylated sequence or a methylated sequence.

In another embodiment, the device may have two or more open reaction chambers and each of the chambers may contain similar material. In another aspect, each of the chambers may have two or more different materials.

In another embodiment, the invention is directed to a method of making a device that may comprise: depositing a reaction mixture comprising a solvent and a reaction material onto the bottom surface of the designated open reaction chamber, near the outlet of the channel connected to said open reaction chamber, and allowing evaporation of solvent and the adsorption of the reaction material to form a pre-deposited solidified material. In yet another aspect, the device may be stored at 4 degrees Celsius until use.

The invention may include a method of analyzing a biological sample using a device comprising: introducing a biological sample through the access point, introducing a immiscible companion fluid to force the biological sample that is inside the channels into the open reaction chambers, allowing the re-suspension of the pre-deposited material, allowing for a biochemical mixture of the biological sample with the re-suspended pre-deposited material, allowing for a biochemical reaction to occur between the biological sample and the re-suspended pre-deposited material, and performing a laboratory diagnostic test of the biochemical mixture. In one aspect, the method may further include introducing a second amount of immiscible companion fluid to prevent possible backflow and cross contamination of said biochemical mixture. In yet another aspect, the biological sample may form droplets in the immiscible companion fluid. In another aspect, the immiscible companion fluid may encapsulate and insulate the biochemical reaction mixture to prevent direct evaporation. In a particular embodiment, the biological sample is purified DNA. In another embodiment, the immiscible companion fluid is immiscible mineral oil. In yet another aspect, the biological sample may be introduced using an injection needle. In one embodiment, the laboratory diagnostic test is DNA methylation detection.

The present invention may be a kit comprising a device, chemical reagents for containing a biological sample, and an immiscible companion fluid. In another aspect, the kit may further include one or more instruments for introducing the biological sample and chemical reagent mixture and the immiscible companion fluid.

### BRIEF DESCRIPTION OF THE DRAWINGS

FIG. 1 shows a device 100 layout having one functional unit 101.

FIG. 2 shows a device 200 layout having nine functional units 201.

FIG. 3 is an illustration of sample loading and primer deposition on an individual functional unit 301.

FIG. 4 is a demonstration of the sample loading scheme with food dye.

FIG. 5 shows the results from the in-tube control reactions.

FIG. 6 shows PCR primers used for MSP. †U represents unmethylated specific primers; ‡M represents methylated primers.

FIG. 7 lays out the principles of methylation Specific PCR (MSP).

### DESCRIPTION

Described herein are devices, assays and methods for analyzing substances.

The present invention provides a device comprising a sampling chamber having an access point and connected to at least one or more channels extending radially outward. Connected to the peripheral or terminal side of the channel is an open reaction chamber.

A device or system as referred to herein may include one or more functional units. A functional unit is the basic unit of the device or system, containing one sampling chamber with one access point and at least one or more channels and at least one or more open reaction chambers. A functional unit may be from about 10 mm to about 100 mm, 10 mm to about 80 mm, 10 mm to about 60 mm, 20 mm to about 60 mm, 20 mm to about 40 mm or 30 mm to about 40 mm in diameter. In a particular embodiment, the functional unit is about 32 mm in diameter. A device may contain multiple functional units e.g., at least about 1, 1 to 50, 5 to 45, 9 to 40, 10 to 30, 15 to 20, or 5, 6, 7, 8, 9, 10, 11, 12, 13, 14 or 15. The device can be made up of a variety of, shapes, e.g., it can be round, ellipse, square, triangle or other irregular shapes. It may be of a variety of sizes, e.g., 1 cm to 50 cm in diameter. The device may comprise a variety of material, e.g., silicon, glass, PMMA, SU-8 or epoxy. In a particular embodiment the device or system may be about 12 cm overall in size.

In a particular embodiment, the device is a microfluidics device, i.e., a reaction vessel comprising at least one microchannel, generally comprising an internal dimension of one millimeter or less. Microfluidics device typically employ very small reaction volumes, often on the order of one or a few microliters, nanoliters (nL), or picoliters (pL). Those in the art will appreciate that the size, shape, and composition of a microfluidics device is generally not a limitation of the current teachings. Rather, a variety of suitable microfluidics devices can be employed in performing one or more steps of the disclosed methods. Descriptions of exemplary microfluidics devices and uses thereof can be found in, among other places, Fiorini and Chiu, BioTechniques 38:429-46, 2005; Kelly and Woolley, Analyt. Chem. 77(5):96A-102A, 2005; Cheuk-Wai Kan et al., Electrophoresis 25:3564-88, 2004; and Yeun et al., Genome Res. 11:405-12, 2001.

A sampling chamber as used herein refers to the area of the device that contains the access point and connects to the channels. This area first houses the introduced sample and may be in a variety of shapes and sizes. For example, it may comprise circles or sectors, and may be from 1 mm to about 10 mm in diameter. In a particular embodiment, the sampling chamber is in the center of the functional unit.

The access point is an aperture, input port or inlet in the sampling chamber that allows the sample to pass into the device. The size of the access point may be from about 0.05 mm to about 5 mm, 0.1 mm to about 4 mm, 0.2 mm to about 3 mm, 0.3 mm to about 2 mm, 0.4 mm to about 1 mm, 0.5 mm to about 0.9 mm, or 0.6 mm to about 0.7 mm in diameter. In a particular embodiment the access point is about 0.64 mm (0.25 inches) in diameter.

The channel may be a micro-channel and are closed units. The channels may be in a variety of shapes and sizes and may be referred to as a microfluidic unit or network. The channel may be from about 20 to about 2000 μm in width. In a particular embodiment the channel is about 500 μm. As another example, the channel cross section may be about 500 μm×50 μm (W×H) and the length may vary. In a particular embodiment, the device may contain from about one to about 50 channels. In a particular embodiment the device contains about six channels. Channels may be branch off or bifurcate into secondary, tertiary or even quaternary channels. A channel may be bifurcated into two or more secondary channels of up to about 50. In a particular embodiment each channel is bifurcated into two secondary channels. Each of the secondary channels may terminate into an open reaction chamber.

The open reaction chamber is an open housing connecting to the peripheral of a channel or a secondary tertiary or even quaternary channel. The open reaction chamber may be a micro-chamber. The open reaction chamber may contain pre-deposited material, will house the reaction after the sample is introduced and may be accessible for surface modification or pre- or post-analysis. The device may contain from about one to about 50 open reaction chambers. In a particular embodiment, the functional unit or device comprises 12 open reaction chambers. The reaction chambers may be equally separated from each other and evenly distributed around the center of the functional unit. The reaction chamber may be of a variety of materials, sizes and shapes. In a particular embodiment, the reaction chamber is oval in shape. The size of the chamber may be from about 0.5 mm to about 20 mm in diameter and may hold up to from about 5 μL to about 2 mL of a solution. The depth of the chamber may be from about 1 mm to 10 mm. There are a variety of manners to make the open reaction chamber, e.g., by punching through-holes with a variety of instruments, e.g., with a hollow puncher.

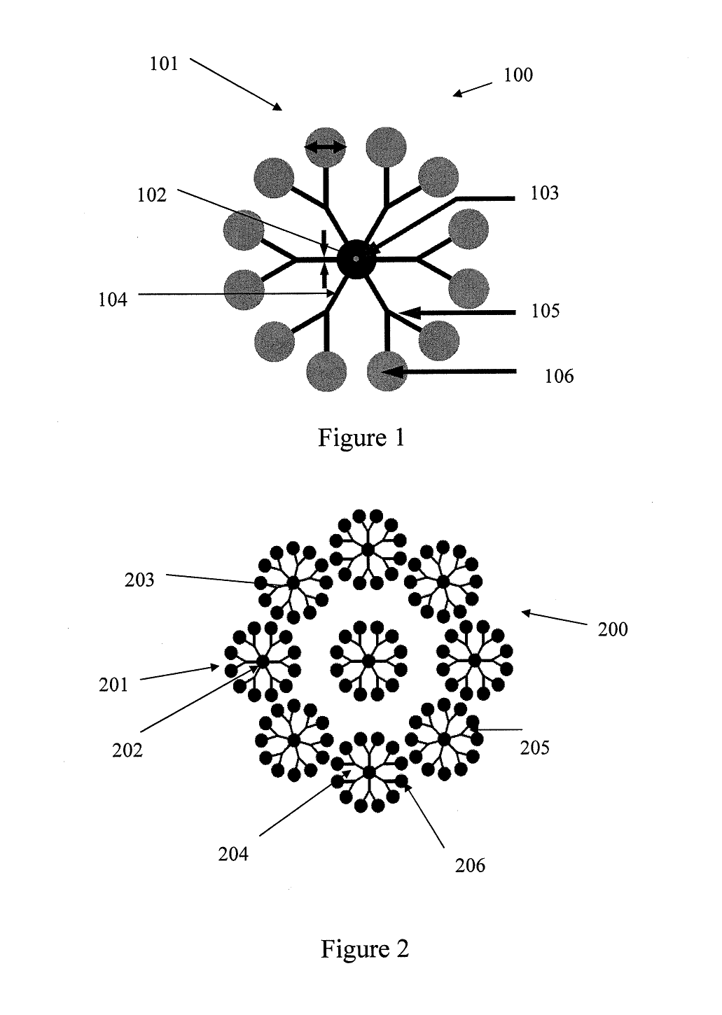

In an example embodiment as shown in FIG. 1, the device 100 comprises one functional unit 101 arranged in a circular, snow flake array, design or pattern. This embodiment has a sampling chamber 102 and access points 103, six channels 104, and twelve secondary channels 105 and twelve open reaction chambers 106. Using such a device, up to twelve different reactions can be prepared with only one sample injection.

In an example embodiment as shown in FIG. 2, the device 200 has nine functional units 201 that are arranged in a circular array. This embodiment has a total of nine sampling chambers 202 and access points 203, fifty-four channels 204, and one hundred and eight secondary channels 205 and one hundred and eight open reaction chambers 206. Using such a device, up to one hundred and eight different reactions can be prepared with only nine sample injections.

The device may be used as an assay or in a system for analyzing substances.

The method, system, laboratory diagnostic test, assay or kit, as used herein refers to a procedure and materials for analyzing, testing and/or measuring the activity of a sample, which contains a substance of interest, e.g., a chemical, a compound, a nucleic acid, an amino acid or an amino acid sequence, a drug, a cell, an enzyme, a protein or other biological component or biochemical in an organism or organic sample. In one embodiment, the device is used for pathogen detection, mutation analysis, and personal genotyping. In a particular embodiment, the sample is a biological sample.

The sample may be the substance of interest in its natural form, isolated and/or modified, and/or may contain one or more buffers, reagents, or other chemicals. In a particular embodiment, the substance is deoxyribonucleic acid (DNA). In another embodiment, the sample is purified DNA. In another embodiment, the purified DNA is subjected to modification such as bisulfite modification. In another embodiment, the purified and modified DNA is placed in an EDTA buffer.

The sample (DNA) amount may be from about 20 pg/μL to about 2 μg/μL.

The term sample is used in a broad sense herein and is intended to include a wide range of biological materials as well as compositions derived or extracted from such biological materials comprising or suspected of comprising gDNA. Exemplary samples include whole blood; nucleated red blood cells; white blood cells; buffy coat; hair; nails and cuticle material; swabs, including buccal swabs, throat swabs, vaginal swabs, urethral swabs, cervical swabs, rectal swabs, lesion swabs, abcess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; pulmonary ravages; lung aspirates; and tissues, including, liver, spleen, kidney, lung, intestine, brain, heart, muscle, pancreas, biopsy material, and the like. The skilled artisan will appreciate that lysates, extracts, or material obtained from any of the above exemplary biological samples are also within the scope of the current teachings. Tissue culture cells, including explanted material, primary cells, secondary cell lines, and the like, as well as lysates, extracts, or materials obtained from any cells, are also within the meaning of the term sample as used herein. Materials comprising or suspected of comprising at least one gDNA target region that are obtained from forensic, agricultural, and/or environmental settings are also within the intended meaning of the term sample. In certain embodiments, a sample comprises a synthetic nucleic acid sequence. In some embodiments, a sample is totally synthetic, for example but not limited to a control sample comprising a buffer solution containing at least one synthetic nucleic acid sequence.

In a particular embodiment, the device is implemented as a versatile miniaturized PCR platform applicable to a PCR-based genomic analysis assay. For example, allele-specific PCR for identifying single nucleotide polymorphisms, asymmetric PCR for generating single stranded DNA hybridization probes or targets, real-time quantitative PCR for DNA/RNA quantification, BEAMing PCR for next-generation sequencing, and methylation-specific PCR (MSP) for DNA methylation detection. In one embodiment, the device is used for DNA methylation detection.

In a particular embodiment, the device may have an open reaction chamber containing a reaction material. In one aspect, the device may be provided without such reaction material, which is provided separately in a container as a liquid or in dehydrated form. The material may be packaged together in a kit with the device or packaged separately. The reaction material may be added to the device before use. There are a variety of devices and instruments that can be used for adding the reaction material, whether before or after packaging. For example the introduction of the reaction mixture be added using a needle, pipette, pump or syringe.

In another embodiment, the device may have open reaction chambers containing the reaction mixture as a pre-deposited solidified material. This device may be packaged and is ready-to-use without further modification or the need to add the material to the open reaction chambers.

A reaction material as used herein refers to a material that will react with the sample to produce a measurable effect. Examples include enzymes, primers, or other material. In a particular embodiment, the reaction material is a PCR primer or a target primer set. In one embodiment, the material may be an unmethylated primer, for example, one specific for a bisulfite modified unmethylated sequence. In another embodiment, the material is a methylated primer, for example, one a specific for bisulfite modified methylated sequence. The material may include primers and probes (e.g. Taqman probe) that target the genes of interest. In yet another aspect, the methylation specific primers are ones that target the promoter regions of two different tumor suppressor gents, e.g., p15 and TMS1.

In another embodiment, the device may have two or more open reaction chambers. The chambers may contain similar material or it may two or more different material. In a particular embodiment the amount of primer material is from about 50 nM to 2 μM after resuspension.

The present invention also provides for a method of analyzing a biological sample using the above described device that contains pre-deposited material in the open reaction chambers. In a particular embodiment the biological sample is introduced into the device via the access point. An immiscible companion fluid is introduced after to force the biological sample in side the channels and into the open reaction chambers.

An immiscible companion fluid as used herein may be immiscible oil, for example, mineral oil, silicon oil, hexadecane or perfluorinated oil. In a particular embodiment the companion fluid is immiscible mineral oil. The immiscible companion fluid may be in an amount of 5 μL to about 100 μL. In a particular embodiment 20 μL is provided. In another embodiment, a second amount of immiscible companion fluid is introduced into the access point to prevent possible backflow and cross contamination of said biochemical mixture.

In a particular embodiment, the biological sample forms droplets in the immiscible companion fluid. In another embodiment, the immiscible companion fluid encapsulates and insulates the material in the open reaction chamber to prevent direct evaporation of any products.

As used here, there are a variety of ways to introduce the sample and/or the companion fluid. The introduction can be made manually by a lab technician using an instrument or by using automated machines. The introduction may be made one at a time or in two or more multiple simultaneous doses, e.g., if more than one functional unit is used. There are a variety of devices and instruments that can be used for introducing the sample. For example the introduction of the sample may be made using a needle, pipette, pump or syringe.

As the aqueous solution of the sample liquid containing the substance of interest and the reagent enters the open reaction chamber, the pre-deposited solid material is re-suspended, and thereby forming a biochemical mixture, which results in a biochemical reaction that occurs between the biological sample and the re-suspended pre-deposited material.

After the reaction, there are a variety of techniques available to analyze the results.

Methylation as used herein refers to the methylation pattern, e.g., methylation of the promoter of the marker, and/or methylation levels of the marker. DNA methylation is a heritable, reversible and epigenetic change. Yet, DNA methylation has the potential to alter gene expression, which has developmental and genetic consequences. DNA methylation has been linked to cancer, as described in, for example, Laird, et al. (1994) Human Molecular Genetics 3:1487-1495 and Laird, P. (2003) Nature 3:253-266, the contents of which are incorporated herein by reference. For example, methylation of CpG oligonucleotides in the promoters of tumor suppressor genes can lead to their inactivation. In addition, alterations in the normal methylation process are associated with genomic instability (Lengauer et al. Proc. Natl. Acad. Sci. USA 94:2545-2550, 1997). Such abnormal epigenetic changes may be found in many types of cancer and can, therefore, serve as potential markers for oncogenic transformation.

Methods for determining methylation include restriction landmark genomic scanning (Kawai et al., Mol. Cell. Biol. 14:7421-7427, 1994), methylation-sensitive arbitrarily primed PCR (Gonzalgo et al., Cancer Res. 57:594-599, 1997); digestion of genomic DNA with methylation-sensitive restriction enzymes followed by Southern analysis of the regions of interest (digestion-Southern method); PCR-based process that involves digestion of genomic DNA with methylation-sensitive restriction enzymes prior to PCR amplification (Singer-Sam et al., Nucl. Acids Res. 18:687, 1990); genomic sequencing using bisulfate treatment (Frommer et al., Proc. Natl. Acad. Sci. USA 89:1827-1831, 1992); methylation-specific PCR (MSP) (Herman et al. Proc. Natl. Acad. Sci. USA 93:9821-9826, 1992); and restriction enzyme digestion of PCR products amplified from bisulfate-converted DNA (Sadri and Hornsby, Nucl. Acids Res. 24:5058-5059, 1996; and Xiong and Laird, Nucl. Acids. Res. 25:2532-2534, 1997); PCR techniques for detection of gene mutations (Kuppuswamyet et al., Proc. Natl. Acad. Sci. USA 88:1143-1147, 1991) and quantitation of allelic-specific expression (Szabo and Mann, Genes Dev. 9:3097-3108, 1995; and Singer-Sam et al., PCR Methods Appl. 1:160-163, 1992); and methods described in U.S. Pat. No. 6,251,594, the contents of which are incorporated herein by reference. An integrated genomic and epigenomic analysis as described in Zardo, et al. (2000) Nature Genetics 32:453-458, may also be used.

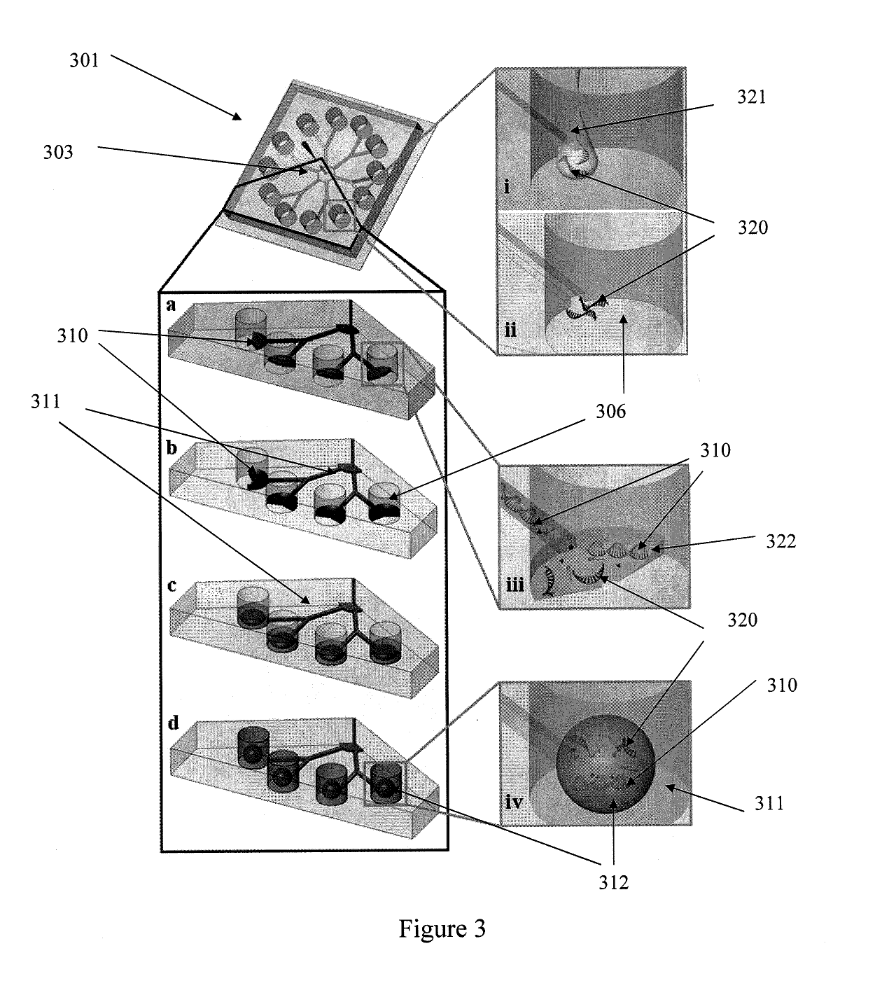

In an example embodiment, as show in FIG. 3, a sample 310 was loaded through access point 303 and to mix with primer deposition 320 on an individual functional unit 301. In FIG. 3a, the sample 310 was injected and guided into each reaction chamber 304. In FIG. 3b, the companion fluid 311 was subsequently injected from the same access point 303, driving the sample 310 into reaction chambers 304. In FIG. 3c, excess companion fluid 311 occupied the residual volume inside the microchannels, preventing possible backflow and cross contamination. In FIG. 3d, the companion fluid 311 filled the entire chamber and covered the sample 310 droplets 312. In FIG. 3i, the primers 320 were deposited onto the bottom of reaction chambers 306. In FIG. 3, ii, the solvent 321 evaporated, leaving primers 321 on the glass substrate. In FIG. 3iii, the resuspension 322 of primers 320 due to infusion of sample 310 containing human gDNA and PCR mix. In FIG. 3iv, all the components required for PCR were trapped inside the droplet 312 that was encapsulated by the companion fluid. The sample 310 is compartmentalized into droplets 312 by the companion fluid 311.

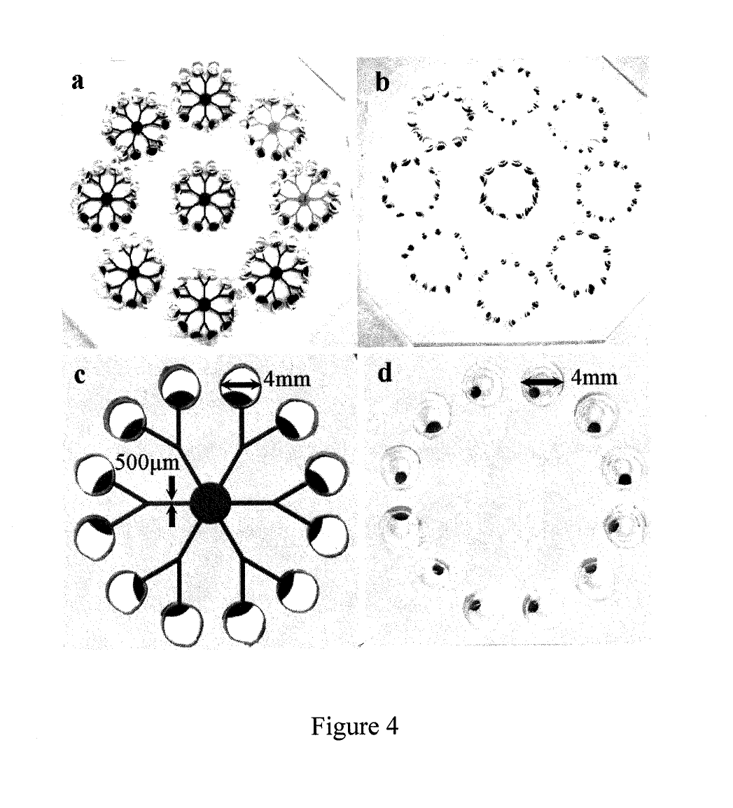

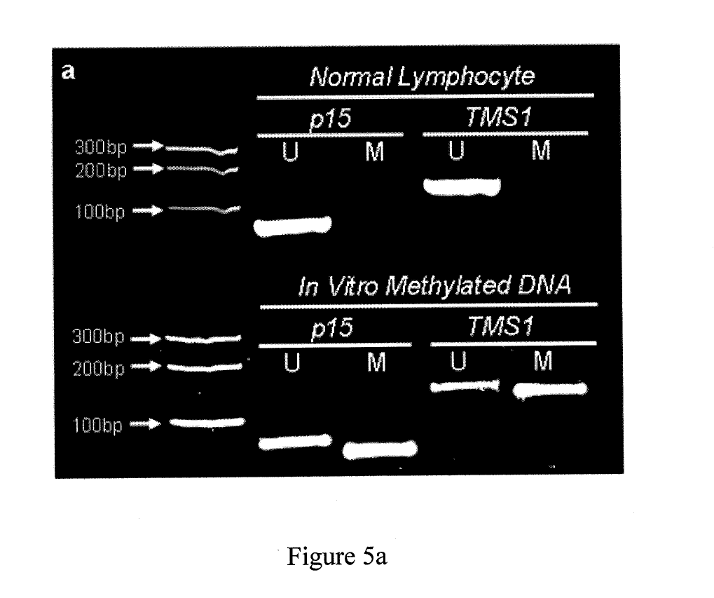

As another example, FIG. 4 is a demonstration of the sample loading scheme with food dye. In FIG. 4a, the entire circular snowflake array was loaded with a total of nine injections. In FIG. 4b, one hundred and eight reactions were prepared after infusion of mineral oil. In FIG. 4c, for each functional unit, 50 μL aqueous food dye was injected from the center sample access port. The dye was guided into twelve reaction chambers through the channels (microfluidic network). In FIG. 4d, the companion fluid forced all the dye into the reaction chambers, leaving no dead volume.

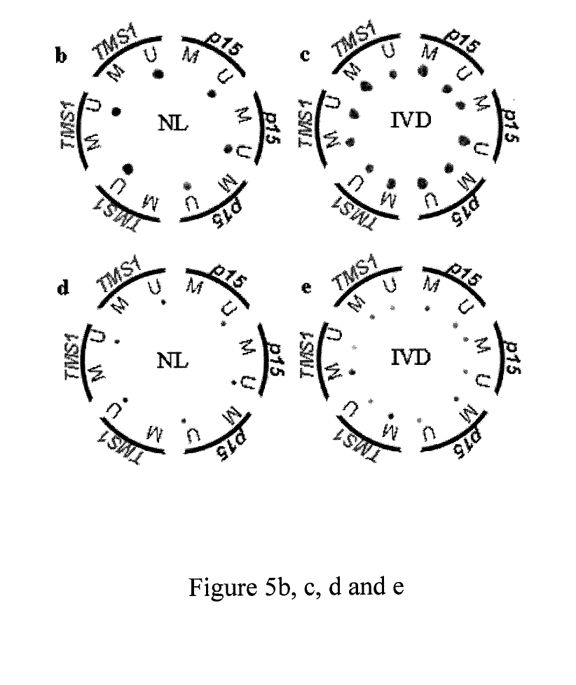

As another example, FIG. 5 shows a comparison of the results between the in-tube control versus the device with open reaction chambers. In FIG. 5a, gel electrophoresis images of in-tube control MSP reactions. Only U primers gave rise to positive amplification in NL sample. M primers resulted in positive amplification with IVD. U bands with IVD sample were due to partial in vitro methylation conversion. Product sizes were 80 by (p15-U), 69 by (p15-M), 140 by (TMS1-U) and 130 by (TMS1-M). b-e) Direct fluorescence images of MSP on chip with dark spots indicating positive amplification. NL sample was only positive with U primers (b,d). IVD sample was positive with both U and M primers due to incomplete conversion (c,e). A total of 50 μL MSP mixture was loaded from the center access port and guided into 12 reaction chambers through the microfluidic network, corresponding to ˜4 μL per reaction (b,c). The open chamber design allowed for flexible reaction volume. A reduced sample, 10 μL, was injected into reaction chambers, which corresponded to ˜0.8 μL per reaction (c, d). The results agreed with control reactions.

In a particular embodiment, the device provides DNA methylation analysis on a droplet-in-oil PCR array. In one aspect, the device is made up of a number of functional units, and provides for a high throughput DNA methylation analysis, utilizing an easy to handle droplet-in-oil microfluidic MSP platform for DNA methylation detection. Methylation specific primers are pre-deposited into the open reaction chambers. The device can perform a large number of MSP reactions in parallel. Each functional unit is capable of DNA methylation analysis of multiple genes with single sample dispensing, thereby significantly reducing the sample preparation time, improving throughput and allowing for automation. In a particular embodiment, the technique uses the mineral oil as a companion fluid for sample actuation, in addition to reaction compartmentalization and evaporation prevention during the thermal cycling. It generates micro droplets by simple injections with a syringe and avoids the use infusion pumps or pressure regulators, making the array an ideal platform for high throughput point-of-care detection.

Further downscaling of sample volume can be performed by using finer needles and syringes for sample injection, yet care needs to be taken to avoid null detection when analyzing the samples containing DNA of very low concentrations.

In another embodiment, present invention provides for an open chamber micro MSP chip that is compatible with conventional bench-top thermal cyclers with minor modification and can be easily be handled by individuals who are not experienced in microfluidics. The device may be portable and is ready-to-use.

Along with chemicals and reagents, the device may be a micro MSP chip(s), which can be provided in the form of diagnostic kits for clinical laboratory applications. In another embodiment, the invention relates to an on-chip DNA methylation analysis using methylation-specific PCR (MSP) within an arrayed micro droplet-in-oil platform that is designed for more practical application of microfluidic droplet technologies in clinical applications. In another aspect, the ready-to-use device includes arrayed primers that are pre-deposited into open micro-reaction chambers and use of the oil phase as a companion fluid for both sample actuation and compartmentalization.

Kits as used herein can include the device described above with one or more of the components for preparing and introducing the sample, e.g., chemical reagents for containing a biological sample or the immiscible companion fluid, or more or more of the components for preparing or coating the reaction material to the open reaction chamber. These components may be packaged together or separately. In a particular embodiment, the kit may include one or more instruments for introducing the biological sample, chemical reagent mixture, or the immiscible companion fluid. In another embodiment, the device may be pre-coated with reaction mixture, e.g., as pre-deposited solid. Such kits, in addition to the containers containing the device or material, optionally include an informational package insert with instructions describing the use and attendant benefits of the components in analyzing material.

Kits can be designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

In certain embodiments, kits comprise a device, a reagent reaction and a target-specific primer pair. In certain embodiments, kits comprise a multiplicity of different target-specific primer pairs.

In a particular embodiment, the devices, compositions, kits, and methods are used for identification, assessment, prevention and therapy of cancer. In yet another embodiment, the described methods and assays may be used for identifying a subject at elevated risk of or having a particular condition, disease or disorder. The individual may be a human or non-human mammal. As used herein, the terms “subject”, “patient”, and “individual” are used interchangeably. In a particular aspect, the invention relates to a method for identifying a subject at elevated risk for an adverse condition, disorder or disease comprising: carrying out one or more assays described above. In a particular embodiment, a biological sample is obtained from the subject and assay is conducting using the device of the present invention.

By “elevated”, “increased” or “high” risk is meant a measurable increase in risk, especially a statistically significant increase in risk, over a control group, e.g. a group of patients having an “average” risk of a particular event, or an elevated risk over the entire population of patients within a particular treatment group.

In a particular embodiment, the condition is cancer. The devices and methods can be used to detect the presence of a cancerous tumor. In a particular embodiment, the device is used to assess DNA methylation to provide early cancer diagnosis and prognosis.

The terms “tumor” or “cancer” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, pancreatic cancer, e.g., pancreatic adenocarcinoma, melanomas, breast cancer, lung cancer, bronchus cancer, colorectal cancer, prostate cancer, pancreas cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like.

In a particular aspect, MSP can be used for analyzing DNA methylation that is closely associated with tumorigenesis, which is a multi-step process resulting from gain-of-function (oncogenes) or loss-of-function (tumor suppressor) gene alterations. These changes could be caused either by genetic or epigenetic changes. Epigenetic changes include the heritable transcriptional silencing of tumor suppressor genes (TSG) by aberrant CpG DNA hypermethylation of their promoters. DNA methylation occurs at 5′ cytosines in CpG di-nucleotides, which when observed in high frequency at many transcription promoter regions and are termed CpG islands. Down regulation of tumor suppressor genes in cancer may be associated with DNA hypermethylation in the promoter regions, where the transcription of DNA to RNA is initiated. The assessment of DNA methylation status can provide a parameter for early cancer diagnosis and prognosis, as well as responsiveness to cancer therapy.

MSP takes advantage of this change in DNA sequence after bisulfite modification, wherein only unmethylated cytosines are converted to uracils, while methylated cytosines remain unaltered. Primers specific to the modified DNA sequences are then introduced to amplify and distinguish methylated from unmethylated DNA. Generally, each bisulfite treated DNA sample is run in two separate MSP reactions. Each reaction contains either a methylated specific primer set or an unmethylated specific primer set, which means the total number of reactions is doubled compared to common PCR based assays.

FIG. 7 lays out the principles of methylation Specific PCR (MSP). Genomic DNA is treated with bisulfite, converting unmethylated cytosines to uracils, while methylated cytosines remains unaltered. Methylated specific (M) primers can only amplify methylated sequence. Unmethylated specific (U) primers can only amplify unmethylated sequence. If M primer is introduced to unmethylated sequence, no amplification is observed due to sequence difference and vice versa.

In some embodiments, the disclosed methods and kits comprise a microfluidics device, “lab on a chip”, or micrototal analytical system (.mu.TAS). In some embodiments, sample preparation is performed using a microfluidics device. In some embodiments, an amplification reaction is performed using a microfluidics device. In some embodiments, a sequencing or Q-PCR reaction is performed using a microfluidic device. In some embodiments, the nucleotide sequence of at least a part of an amplification product is obtained using a microfluidics device. Descriptions of exemplary microfluidic devices can be found in, among other places, Published PCT Application Nos. WO/0185341 and WO 04/011666; Kartalov and Quake, Nucl. Acids Res. 32:2873-79, 2004; and Fiorini and Chiu, BioTechniques 38:429-46, 2005.

In certain embodiments, the disclosed methods and kits comprise a solid support. Non-limiting examples of solid supports include, agarose, sepharose, polystyrene, polyacrylamide, glass, membranes, silica, semiconductor materials, silicon, organic polymers; optically identifiable micro-cylinders; biosensors comprising transducers; appropriately treated or coated reaction vessels and surfaces, for example but not limited to, micro centrifuge or reaction tubes, wells of a multiwell microplate, and glass, quartz or plastic slides and/or cover slips; and beads, for example but not limited to magnetic beads, paramagnetic beads, polymer beads, metallic beads, dye-impregnated or labeled beads, coated beads, glass beads, microspheres and nanospheres. In some embodiments, a solid support is used in a separating and/or detecting step, for example but not limited to, for purifying and/or analyzing amplification products. Those in the art will appreciate that any number of solid supports may be employed in the disclosed methods and kits and that the shape and composition of the solid support is generally not limiting.

A variety of methods are available for obtaining gDNA for use with the current teachings. Methylated and unmethylated gDNA is also commercially available. When the gDNA is obtained through isolation from a biological matrix, preferred isolation techniques include (1) organic extraction followed by ethanol precipitation, e.g., using a phenol/chloroform organic reagent (see, e.g., Sambrook et al.; Ausubel et al.), for example using an automated DNA extractor, e.g., the Model 341 DNA Extractor (Applied Biosystems, Foster City, Calif.); (2) stationary phase adsorption methods (e.g., Boom et al., U.S. Pat. No. 5,234,809; Walsh et al., Biotechniques 10(4): 506-513, 1991); and (3) salt-induced DNA precipitation methods (see, e.g., Miller et al., Nucl. Acids Res. 16(3): 9-10, 1988), such precipitation methods being typically referred to as “salting-out” methods. In certain embodiments, gDNA isolation techniques comprise an enzyme digestion step to help eliminate unwanted protein from the sample, for example but not limited to, digestion with proteinase K, or other like proteases; a detergent; or both (see, e.g., U.S. Patent Application Publication 2002/0177139; and U.S. patent application Ser. Nos. 09/724,613 and 10/618,493). Commercially available nucleic acid extraction systems include, among others, the ABI PRISM® 6100 Nucleic Acid PrepStation and the ABI PRISM® 6700 Nucleic Acid Automated Work Station; nucleic acid sample preparation reagents and kits are also commercially available, including, NucPrep™ Chemistry, BloodPrep™ Chemistry, the ABI PRISM® TransPrep System, and PrepMan™ Ultra Sample Preparation Reagent (all from Applied Biosystems).

According to the instant teachings, gDNA may be obtained from any living, or once living, organism, including a prokaryote, an archaea, or a eukaryote, for example but not limited to, an insect including Drosophila, a worm including C. elegans, a plant, and an animal, including a human; and including prokaryotic cells and cells, tissues, and organs obtained from a eukaryote, for example but not limited to, cultured cells and blood cells. Certain viral genomic DNA is also within the scope of the current teachings. In certain embodiments, the gDNA may be present in a double-stranded or single-stranded form. The skilled artisan appreciates that gDNA includes not only full length material, but also fragments generated by any number of means, for example but not limited to, enzyme digestion, sonication, shear force, and the like, and that all such material, whether full length or fragmented, represent forms of gDNA that can serve as templates for an amplifying reaction of the current teachings.

According to the disclosed methods, a sample comprising at least one gDNA target region is exposed to a modifying agent and a modified sample is obtained. The term “modified sample” refers to a sample that has been exposed to a modifying agent under conditions suitable for the modifying agent to generate at least one modified nucleotide. Typically the modifying agent will interact with or convert at least one target nucleotide in the gDNA to generate at least one modified nucleotide. Non-limiting examples of compounds that may serve as suitable modifying agents include bisulfite compounds, for example but not limited to, sodium bisulfite, magnesium bisulfite, manganese bisulfite, potassium bisulfite, ammonium bisulfite; 5-bromouracil; and certain sulfhydryl compounds, for example but not limited to, mercaptoethanol, cysteine methyl ester, glutathione, and cysteamine. Descriptions of exemplary modifying agents can be found in, among other places, Hayatsu, Prog. Nucl. Acid Res. Mol. Biol. 16:75-124, 1975; Hayatsu, Proc. Japanese Acad. Ser. B, 80:189-94, 2004; Boyd and Zon, Anal. Biochem. 326:278-80, 2004; U.S. patent application Ser. No. 10/926,530; and U.S. Published Patent Appl. No. US 2005-008989A1).

In certain embodiments, a sample comprising gDNA is treated with the modifying agent sodium bisulfite, which converts unmethylated cytosines (“target nucleotides”) to uracil (the “modified nucleotide”), while methylated cytosines (also target nucleotides) are generally non-reactive. At least one target region in the bisulfite treated gDNA is amplified, typically by PCR using target-specific primers to yield first amplicons in which uracil residues are converted to thymine, while methylated cytosine is amplified as cytosine. In the case of samples comprising mixed cell populations, for example but not limited to tumor biopsy samples containing both normal cells and cancerous cells, the cytosine content of the amplified DNA from the various cell subpopulations can be very different, with unmethylated DNA being T-rich and C-deficient after conversion, while the amplicons from methylated target regions can retain at least some of the original cytosine content.

Those in the art will appreciate that treatment of gDNA with certain modifying agents, for example but not limited to sodium bisulfite, cause unmethylated C to be deaminated to U. A gDNA flanking region comprising an unmethylated C would, after sodium bisulfite treatment, result in a modified nucleotide in the flanking region of modified sample, which could prevent or decrease the ability of the corresponding target-specific primer to selectively hybridize. The target-specific primers of the current teachings are typically designed to selectively hybridize with target flanking sequences that are outside CpG islands to allow a target region amplicon to be generated regardless of the methylation state of the target region.

In certain embodiments, a gDNA sample comprising at least one target region is treated with a modifying agent to obtain a modified sample comprising at least one modified target nucleotide. The term “modifying agent” refers to any reagent that can modify a nucleic acid, for example but not limited to at least one target nucleotide in at least one gDNA target region. Some modifying agents convert an unmethylated target nucleotide to a modified nucleotide, but do not convert a methylated target nucleotide to a modified nucleotide (at least not to a significant degree).

In certain embodiments, bisulfite is employed as a modifying agent. Incubating nucleic acid sequences such as gDNA with bisulfite results in deamination of a substantial portion of unmethylated cytosines, which converts such cytosines to uracil. Methylated cytosines are deaminated to a measurably lesser extent. In certain embodiments, the sample is then amplified, resulting in the uracil bases being replaced with thymine. Thus, in certain embodiments, a substantial portion of unmethylated target cytosines ultimately become thymines, while a substantial portion of methylated cytosines remain cytosines. In certain embodiments, the presence of a modified nucleotide (for example but not limited to, uracil or thymine) in the target region may be determined using the methods and kits of the present teachings. Descriptions of bisulfite treatment can be found in, among other places, U.S. Pat. Nos. 6,265,171 and 6,331,393; Boyd and Zon, Anal. Biochem. 326: 278-280, 2004; U.S. Provisional Patent Application Ser. Nos. 60/499,113; 60/520,942; 60/499,106; 60/523,054; 60/498,996; 60/520,941; 60/499,082; and 60/523,056.

The skilled artisan will appreciate that the complement of the disclosed gDNA target regions, primers, target-specific portions, primer-binding sites, or combinations thereof, may be employed in certain embodiments of the present teachings.

The term “primer” refers to a polynucleotide that selectively hybridizes to a gDNA target flanking sequence or to a corresponding primer-binding site of an amplification product; and allows the synthesis of a sequence complementary to the corresponding polynucleotide template from its 3′ end.

A “target-specific primer pair” of the current teachings comprises a forward target-specific primer and a reverse target-specific primer. The forward target-specific primer comprises a first target-specific portion that comprises a sequence that is the same as or substantially the same as the nucleotide sequence of the first or upstream target flanking sequence, and that is designed to selectively hybridize with the complement of the upstream target flanking sequence that is present in, among other places, the reverse strand amplification product.

In some embodiments, a multiplicity of different primer pairs are employed in an amplifying step, for example but not limited to a multiplex amplification reaction, wherein the different primer pairs are designed to amplify a multiplicity of different nucleotide sequences, including a multiplicity of different gDNA target regions or a multiplicity of different amplification products.

The sequence-specific portions of the disclosed primers are of sufficient length to permit specific annealing with complementary or substantially complementary sequences in target flanking sequences and/or amplicons, as appropriate. The criteria for designing sequence-specific primers are well known to persons of ordinary skill in the art. Descriptions of primer design can be found in, among other places, Diffenbach and Dveksler, PCR Primer, A Laboratory Manual, Cold Spring Harbor Press (1995); Rapley, The Nucleic Acid Protocols Handbook (2000), Humana Press, Totowa, N.J. (hereinafter “Rapley”); and Kwok et al., Nucl. Acid Res. 18:999-1005 (1990).

The skilled artisan will appreciate that while the primers and primer pairs of the present teachings may be described in the singular form, a plurality of primers may be encompassed by the singular term. Thus, for example, in certain embodiments, a target-specific primer pair typically comprises a plurality of forward target-specific primers and a plurality of corresponding reverse target-specific primers.

Those in the art understand that primers and primer pairs that are suitable for use with the disclosed methods and kits can be identified empirically using the current teachings and routine methods known in the art, without undue experimentation.

The terms “amplifying” and “amplification” are used in a broad sense and refer to any technique by which a target region, an amplicon, or at least part of an amplicon, is reproduced or copied (including the synthesis of a complementary strand), typically in a template-dependent manner, including a broad range of techniques for amplifying nucleic acid sequences, either linearly or exponentially. Some non-limiting examples of amplification techniques include primer extension, including the polymerase chain reaction (PCR), RT-PCR, asynchronous PCR (A-PCR), and asymmetric PCR, strand displacement amplification (SDA), multiple displacement amplification (MDA), nucleic acid strand-based amplification (NASBA), rolling circle amplification (RCA), transcription-mediated amplification (TMA), and the like, including multiplex versions and/or combinations thereof. Descriptions of certain amplification techniques can be found in, among other places, Molecular Cloning, A Laboratory Manual, Cold Spring Harbor Press, 3d ed., 2001 (hereinafter “Sambrook and Russell”); Sambrook et al.; Ausubel et al.; PCR Primer: A Laboratory Manual, Diffenbach, Ed., Cold Spring Harbor Press (1995); Msuih et al., J. Clin. Micro. 34:501-07 (1996); McPherson; Rapley; U.S. Pat. Nos. 6,027,998 and 6,511,810; PCT Publication Nos. WO 97/31256 and WO 01/92579; Ehrlich et al., Science 252:1643-50 (1991); Favis et al., Nature Biotechnology 18:561-64 (2000); Protocols & Applications Guide, rev. 9/04, Promega, Madison, Wis.; and Rabenau et al., Infection 28:97-102 (2000).

The terms “amplification product” and “amplicon” are essentially used interchangeably herein and refer to the nucleic acid sequences generated from any cycle of amplification of any amplification reaction, for example a first amplicon is generated during a first amplification reaction and a second amplicon product is generated during a second amplification reaction, unless otherwise apparent from the context. An amplicon can be either double-stranded or single-stranded, including the separated component strands obtained from a double-stranded amplification product.

In certain embodiments, amplification techniques comprise at least one cycle of amplification, for example, but not limited to, the steps of: selectively hybridizing a primer to a target region flanking sequence or a primer-binding site of an amplicon (or complements of either, as appropriate); synthesizing a strand of nucleotides in a template-dependent manner using a polymerase; and denaturing the resulting nucleic acid duplex to separate the strands. The cycle may or may not be repeated.

In some embodiments, the methods of the current teachings are performed before, after, or in conjunction with a Q-PCR reaction. The term “quantitative PCR”, or “Q-PCR”, refers to a variety of methods used to quantify the results of the polymerase chain reaction for specific nucleic acid sequences. Such methods typically are categorized as kinetics-based systems, that generally determine or compare the amplification factor, such as determining the threshold cycle (C.sub.1), or as co-amplification methods, that generally compare the amount of product generated from simultaneous amplification of target and standard templates. Many Q-PCR techniques comprise reporter probes, intercalating agents, or both. For example but not limited to TaqMan® probes (Applied Biosystems), i-probes, molecular beacons, Eclipse probes, scorpion primers, Lux™ primers, FRET primers, ethidium bromide, SYBR® Green I (Molecular Probes), and PicoGreen® (Molecular Probes).

The term “analyzing” when used in reference to amplification or a first amplicon, part of a first amplicon, a second amplicon, part of a second amplicon, or combinations thereof, includes any technique that allows one or more parameter of an amplicon or at least part of an amplicon to be obtained. In certain embodiments, analyzing comprises (1) separating (at least partially) one amplicon species from another amplicon species, including amplicons derived from different target regions and amplicons derived from the same target region but with different degrees of methylation (e.g., fully methylated, unmethylated, and intermediate levels of methylation, sometimes referred to as a group or family of “related amplicons”), (2) detecting a separated and/or partially separated amplicon, and (3) obtaining and evaluating one or more amplicon parameter, for example but not limited to, amplicon peak height, integrated area under an amplicon peak, and amplicon intensity, including the fluorescent intensity of an incorporated fluorescent reporter group, the luminescent intensity of an incorporated bioluminescent, chemiluminescent, and/or phosphorescent reporter group, and the radioactive intensity of an incorporated isotope. Typically, one or more parameter(s) of one amplicon is compared with the same parameter(s) of another amplicon to determine the degree of target region methylation, including qualitative, semi-quantitative, and quantitative determinations. The degree of methylation of at least one target region is typically determined by inference, for example but not limited to, by determining whether an amplicon derived from a modified sample comprises a modified nucleotide or its complement and inferring that the corresponding target region is methylated or is not methylated.

Non-limiting examples of methylation detection and/or quantitation methods that may be employed before, after, and/or in conjunction with certain disclosed methods include melting curve analysis of modified and/or unmodified target regions, methylation specific PCR (MSP), MethyLight, methyl-acceptor assay, enzymatic regional methylation assay (ERMA), certain assays using methylation-sensitive and insensitive restriction endonucleases, for example but not limited to the HpaII/MspI isoschizomer pair, combined bisulfite restriction analysis (COBRA), hydrazine and/or permanganate treatment with or without ligation-mediated PCR, restriction landmark genomic scanning (RLGS), differential methylation hybridization, methylation-specific oligonucleotide microarray technique, methylation assay by nucleotide incorporation (MANIC), methylation-sensitive single nucleotide primer extension (Ms-SnuPE), pyrosequencing methylation analysis (PyroMethA), DHPLC-based DNA methylation analysis; and bisulfate genomic sequencing (see, e.g., Herman et al., Proc. Natl. Acad. Sci. 93:9821-26, 1996; Yamamoto et al., BioTechniques 36(5):846-54, 2004; Eads et al., Nucl. Acids Res. 28(8):e32, 2000; Collela et al., BioTechniques, 35(1):146-50, 2003; Xiong and Laird, Nucl. Acids Res. 25(12):2532-34, 1997; Granau et al., Nucl. Acids Res., 29(13):e65, 2001; Convert et al., BioTechniques 34(2):356-62, 2003; and Kupper et al., BioTechniques 23(5):843-46, 1997).

In some embodiments, a primer and/or an amplification product comprise an affinity tag. In some embodiments, an affinity tag comprises a reporter group. In certain embodiments, affinity tags are used for separating, are part of a detecting means, or both. In some embodiments, a primer and/or amplification product comprises a hybridization tag, a hybridization tag complement, or both. The term “reporter probe” refers to a sequence of nucleotides, nucleotide analogs, or nucleotides and nucleotide analogs, that binds to or anneals with an amplicon, and when detected, including a change in intensity or of emitted wavelength, is used to identify and/or quantify the corresponding amplicon. In certain embodiments, a reporter probe comprises a fluorescent reporter group, a quencher reporter group (including dark quenchers and fluorescent quenchers), an affinity tag, a hybridization tag, a hybridization tag complement, or combinations thereof.

Those in the art appreciate that as an amplification product is amplified by certain amplification techniques, the complement of the primer-binding site is synthesized in the complementary strand. Thus, it is to be understood that the complement of a primer-binding site is expressly included within the intended meaning of the term primer-binding site, unless stated otherwise.

As used herein, the term “degree of methylation” when used in reference to a gDNA target region, refers to the amount of that target region within a sample that is methylated relative to the amount of the same target region that is not methylated, or to the relative number of methylated nucleotides in a target region, or both. In certain embodiments, a sample contains a target region that is fully methylated, a target region that is unmethylated, a target region that has some copies that are fully methylated and some copies that are unmethylated.

The term “DNA polymerase” is used in a broad sense herein and refers to any polypeptide that is able to catalyze the addition of deoxyribonucleotides or analogs of deoxyribonucleotides to a nucleic acid polymer in a template dependent manner. Typically DNA polymerases include DNA-dependent DNA polymerases and RNA-dependent DNA polymerases, including reverse transcriptases.

The term “reporter group” is used in a broad sense herein and refers to any identifiable tag, label, or moiety. The skilled artisan will appreciate that many different species of reporter groups can be used in the present teachings, either individually or in combination with one or more different reporter group.

For the purposes of this definition, the term “sequence” includes nucleic acid sequences, polynucleotides, oligonucleotides, primers, target-specific portions, amplification product-specific portions, primer-binding sites, hybridization tags, and hybridization tag complements.

The term “target region” may refer to the gDNA segment that is being amplified and analyzed to determine the presence or absence of methylated nucleotides and infer the degree of target region methylation. A target region may be located in the promoter or regulatory elements of a gene of interest that is known or suspected of being methylated under certain physiological conditions. The target region is generally located between two flanking sequences, a first target flanking region and a second target flanking region, located on either side of, but not necessarily immediately adjacent to, the target region. In some embodiments, a gDNA segment comprises a plurality of different target regions. In some embodiments, a target region is contiguous with or adjacent to one or more different target regions. In some embodiments, a given target region can overlap a first target region on its 5′-end, a second target region on its 3′-end, or both.

A target region can be either synthetic or naturally occurring. Certain target regions, including flanking sequences where appropriate, can be synthesized using oligonucleotide synthesis methods that are well-known in the art.

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H.sup.+, NH.sub.4.sup.+, trialkylammonium, Mg.sup.2+, Na.sup.+, and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytosine or possibly 5-methyldeoxycytosine (5 mC), “G” denotes deoxyguanosine, “T” denotes thymidine, and “U” denotes deoxyuridine, unless otherwise noted.

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including patents, patent applications, articles, books, treatises, and internet web pages are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The term “at least some of”, for example, when used in reference to a sample or a modified sample, means that all of the sample or modified sample can be used or that some, but not all, of the sample or modified sample can be used, for example, an aliquot. The term “at least part of”, for example, when used in reference to analyzing an amplification product, means that the entire amplification product can be analyzed, one or both of the individual strands of a double-stranded amplification product can be analyzed, or a fragment, portion, or subsequence of an amplification product can be analyzed.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “corresponding” as used herein refers to at least one specific relationship between the elements to which the term relates.

As used herein, the singular forms “a”, “an”, and “the” include plural forms unless the context clearly dictates otherwise. Thus, for example, reference to “an assay” includes a plurality of such assay.

The invention is to be understood as not being limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein, including patents, patent applications, and journal articles are incorporated herein by reference in their entireties including the references cited therein, which are also incorporated herein by reference.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The invention is to be understood as not being limited to particular embodiments described, as such may, of course, vary. It is also to be understood that the terminology used herein is for the purpose of describing particular embodiments only, and is not intended to be limiting, since the scope of the present invention will be limited only by the appended claims.

### EXAMPLES

### Example 1

### DNA Methylation Analysis on a Droplet-In-Oil PCR Array

### Summary

On-chip DNA methylation analysis was performed using methylation-specific PCR (MSP) within an arrayed micro droplet-in-oil platform that is designed for more practical application of microfluidic droplet technologies in clinical applications. Unique features of this ready-to-use device include arrayed primers that are pre-deposited into open micro-reaction chambers and use of the oil phase as a companion fluid for both sample actuation and compartmentalization. These technical advantages allow for infusion of minute amounts of sample for arrayed MSP analysis, without the added complexities inherent in microfluidic droplet-based studies. Ease of use of this micro device is exemplified by analysis of two tumor suppressor promoters, p15 and TMST using an on-chip methylation assay. These results were consistent with standard MSP protocols, yet the simplicity of the droplet-in-oil microfluidic PCR platform provides and easy and efficient tool for DNA methylation analysis in a large-scale arrayed manner.

### Introduction

MSP (FIG. 7) is most extensively used for analyzing DNA methylation that is closely associated with tumorigenesis, a multi-step process resulting from gain-of-function (oncogenes) or loss-of-function (tumor suppressor) gene alterations. These changes could be caused either by genetic or epigenetic changes. One of the most well studied epigenetic changes is the heritable transcriptional silencing of tumor suppressor genes (TSG) by aberrant CpG DNA hypermethylation of their promoters. In higher order eukaryotes, DNA methylation only occurs at 5′ cytosines in CpG dinucleotides, which when observed in high frequency at many transcription promoter regions and are termed CpG islands. There is clear evidence that the down regulation of tumor suppressor genes in cancer is tightly associated with DNA hypermethylation in the promoter regions, where the transcription of DNA to RNA is initiated. Therefore, the assessment of DNA methylation status has great clinical implications, offering another important parameter for early cancer diagnosis and prognosis, as well as responsiveness to cancer therapy. MSP takes advantage of this change in DNA sequence after bisulfite modification, wherein only unmethylated cytosines are converted to uracils, while methylated cytosines remain unaltered. Primers specific to the modified DNA sequences are then introduced to amplify and distinguish methylated from unmethylated DNA. Generally, each bisulfate treated DNA sample is run in two separate MSP reactions. Each reaction contains either a methylated specific primer set or an unmethylated specific primer set, which means the total number of reactions is doubled compared to common PCR based assays. In a single DNA methylation study of clinical samples, hundreds or even thousands of analyses are carried out at different promoter regions. MSP assays with such a great number of samples are cumbersome and can often take months. Therefore, a higher throughput approach would be desirable.

To address the increasing interest in high throughput DNA methylation analysis, an easy to handle droplet-in-oil microfluidic MSP platform was developed for DNA methylation detection. The device has 9 functional snowflake-like units arranged in a circular array. Each functional unit consists of 12 open reaction chambers which are also arranged in a circular array and connected to sample access port through a microfluidic network. Methylation specific primers are pre-deposited into reaction chambers. The device can perform 108 MSP reactions in parallel. Each functional unit is capable of DNA methylation analysis of multiple genes with single sample dispensing, thereby significantly reducing the sample preparation time, improving throughput and allowing for automation. The simple and disposable device can easily be handled by individuals who do not have microfluidic experience because it takes the advantage of a novel sample loading scheme. The unique scheme uses the mineral oil as a companion fluid for sample actuation, in addition to reaction compartmentalization and evaporation prevention during the thermal cycling. It generates micro droplets by simple injections with a syringe and avoids the use infusion pumps or pressure regulators, making the array an ideal platform for high throughput point-of-care detection. The MSP device was validated by testing with multiple gene analyses and comparing the result with conventional in-tube MSP assay.

### Device Design, Fabrication and Preparation

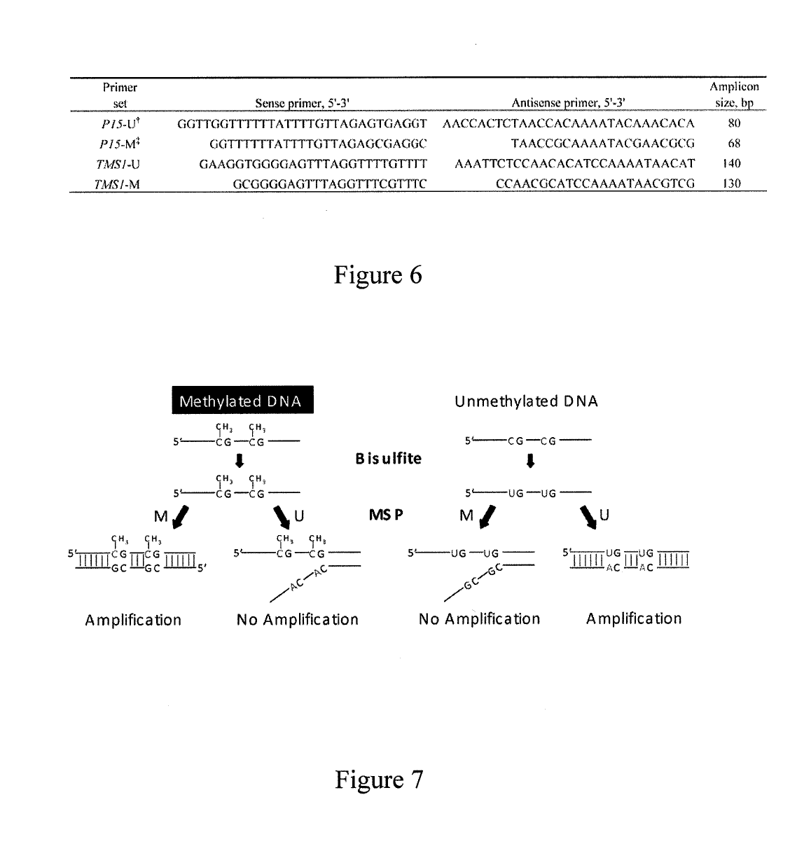

The device contains 9 functional units arranged in a circular array (FIG. 2a). The layout of each functional unit resembles a snowflake where 6 channels of 500 μm width extend from a center sample input port to peripheral reaction chambers (FIG. 2b). Each channel was symmetrically bifurcated and terminated at two different chambers. The 12 reaction chambers were equally separated from each other and evenly distributed around the center circle. The size of each functional unit is 32 mm in diameter and the overall size of the system is 12 cm. The prototype device was fabricated by casting polydimethylsiloxane (PMDS) (Corning Inc.) over a lithographically patterned 50 μm thick SU-8 master on silicon substrate. The sample access port was created by punching a through-hole at the origin of the center circle with a dispensing needle of 0.64 mm (0.25 inch) diameter. The reaction chambers were made by punching through the peripheral circles using a hollow puncher of 4 mm diameter. The punched PDMS was then bonded to a thin glass slide (Fisher Scientific Inc.) after oxygen plasma treatment. Bonded chips were baked at 75° C. overnight. To prevent possible enzyme adsorption and improve PCR efficiency, the surface of the reaction chambers was dip coated with 0.1% (wt %) Bovine Serum Albumin (BSA). Methylation specific primers (FIG. 6) that target the promoter regions of two different tumor suppressor genes (p15 and TMS1) were pre-deposited into designated reaction chambers. Primer deposition was accomplished by pre-metering primers and allowing full-solvent evaporation on the bottom surface of the chambers. The primer deposition process could be automated using inkjet technology. 39 Prepared chips were thus primed for simultaneous methylation detection and stored at 4° C. for future use.

### DNA Isolation and Bisulfite Modification

Human genomic DNA (gDNA) was extracted from human peripheral blood, which was drawn from a volunteer after the receipt of informed consent. All chemicals used in the sample preparation were purchased from Sigma Aldrich Inc. unless otherwise stated. Normal leukocytes (NL) were extracted by centrifugation. The extract was digested by 0.5 mg/mL proteinase K in 50 μL PK buffer, 19.5 mM ammonium sulfate, 78.8 mM Tris, 7.9 mM magnesium chloride and 11.7 nM 2-mercaptoethnoal at 60° C. Proteinase K was then inactivated by heating to 100° C. for 10 minutes. DNA was purified by phenol-chloroform extraction. In vitro methylated DNA (IVD) was obtained by treating NL DNA with SSSI methyltransferase and purified using Wizard DNA CleanUp System (Promega). The purified DNA was subjected to bisulfite modification. 1 μg DNA was first denatured by adding NaOH (final concentration of 0.2M) and incubation at 37° C. for 10 minutes. 520 μL of sodium bisulfite (concentration of 3M) and 30 μL of hydroquinone (concentration 10 mM), freshly prepared before each bisulfite treatment, were added to the denatured DNA, gently vortexed and incubated at 55° C. for 16 hours. Subsequently, the bisulfite-modified DNA was purified using Wizard DNA CleanUp System (Promega) following the manufacturer's instructions and eluted into 50 μL water. Lastly, the purified DNA was incubated with 0.3 M NaOH at room temperature, followed by ethanol precipitation. The final modified DNA product was suspended in 20 μL of Tris EDTA buffer (pH 8.5) and used immediately or kept at −20° C. for long term storage.

### Methylation-Specific Polymerase Chain Reaction (MSP) Protocol

As way of background, unmethylated cytosines are chemically converted to uracils by bisulfite treatment, whereas methylated cytosines remain as cytosines. The sequence difference between the methylated and unmethylated DNA after bisulfite modification are recognized and amplified by methylation specific primers. Primer sequences are selected from regions containing CpG islands, thus providing maximal discrimination between methylated and unmethylated DNA.

The primer pairs as used herein are listed in FIG. 6. A total of two sets of four primer pairs were purchased from the Integrated DNA Technology (IDT DNA). Each set includes unmethylated (U) and methylated (M) primers which are specific for bisulfite modified unmethylated sequence and methylated sequence respectively. M primers can only amplify the bisulfite modified methylated sequence and vice versa. The MSP mixture (50 μl) contains dNTPs (each at 1.5 mM), 1×PCR buffer (67 mM Tris, 16.6 mM ammonium sulfate, 6.7 mM MgCl2, 10 nM 2-mercaptoethanol), 0.05 U/μL HotStar Taq polymerase (Qiagen Inc.), 1×DNA intercalating dye EvaGreen (Biotium Inc.), 0.1% (wt %) BSA, bisulfite treated DNA as template, and PCR grade water to adjust the final volume. The thermal cycling conditions are as follows: enzyme activation and pre-denaturation at 95° C. for 15 min, followed by 30 cycles of denaturation at 95° C. for 30 s, annealing at 60° C. for 30 s and elongation at 72° C. for 30 s. A final elongation step is performed at 72° C. for 5 min.

Conventional MSP assays were performed using NL (unmethylated DNA sample) and IVD (methylated DNA sample) as templates. Four pairs of primers, namely p15-U, p15-M, TMS1-U and TMS1-M (FIG. 6) were used to assess the methylation status of the DNA samples. Reactions were carried out in conventional thermal cycler (Bio-Rad Inc.) with 50 μL reaction volume. Final MSP products were loaded onto 2% agarose gel, stained with GelStar® nucleic acid gel stain (Lonza Group Ltd.) and imaged with UV illumination system. On-chip MSP reactions were performed under the identical condition as conventional MSP reactions. MSP mixtures also contained 0.1% (wt %) BSA as a dynamic coating against enzyme adsorption. EvaGreen served as a fluorescence reporter for imaging purposes. MSP mixture was loaded onto the chips using an injection needle. The total amount of pre-deposited primers in each reaction chamber was metered according to the final volume, so that the final primer concentration was 300 nM. The thermal cycling was performed with a flat-bed peltier heating plate. The MSP chip was thermally coupled to the heating plate by mineral oil and slightly pressed against the heating plate to ensure full contact. The direct fluorescence image of the micro MSP chip was taken using a Typhoon™ scanner (GE Healthcare) with 488 nm excitation. The fluorescence emission was filtered by a band pass filter, which centers at 520 nm with a bandwidth of 40 nm, before it was collected. The gain of the internal photomultiplier tube was set to 230 with normal sensitivity. Images were processed with ImageJ (NIH) to remove background noise and enhance contrast.

### Results and Discussion

The sample loading scheme is demonstrated in FIG. 3. Primers were deposited onto the bottom surface of the designated reaction chambers, near the outlets of the microchannels connected to the reaction chambers, during the device fabrication and preparation stage. Oligonucleotides were adsorbed to the surface after full solvent evaporation. The chips were stored at 4° C. until use. For each functional unit, first the sample was injected from the center sample access port through a dispensing needle. The sample liquid was guided to the reaction chambers through the microfluidic network (FIG. 3a). The aqueous solution dissolved the primers, thus completing the ingredient list of the MSP mixture. Because each reaction chamber contains a unique set of primers, multiple MSP reactions could be prepared with single sample injection. For the current circular snowflake array, only 9 injections were required for preparing 108 distinct MSP reactions, which significantly reduced the sample preparation time and improved the throughput. Next, metering and compartmentalization were accomplished using immiscible mineral oil as companion fluid. The mineral oil forced the sample inside the microchannels into reaction chambers, allowing resuspension and mixing of the pre-deposited primers with the DNA sample and MSP reagent (FIG. 3b). Excess oil was then injected to fill the volume in the microchannels and prevent possible backflow and cross contamination (FIG. 3c). Finally, the MSP mixture formed droplets in the mineral oil and sat on the bottom of the reaction chambers due to differences in solvent polarities and densities. Consequently, the oil provided insulation for the reaction chambers against direct evaporation (FIG. 3d). To aid visualization, this sample loading scheme was monitored using aqueous food dye. Pictures of the device with food dye before and after the infusion of the mineral oil are shown in FIG. 4.

This sample loading scheme has several advantages. First, multiple reactions are prepared with a single sample injection, which improves the throughput and allows for automation. Second, since eventually all the sample will be pushed into each reaction chamber, there is no dead volume, enabling the loading of minute amounts of sample. Last, unlike other micro PCR arrays, the reaction volume of the droplet-in-oil MSP chip is not defined by the size of the reaction chamber, and, thus the assay is more flexibility in terms of reaction volume. MSPs with different reaction volumes can be performed on the same device.

The gel image of the conventional reactions (FIG. 5a) revealed the methylation status of the input DNA templates. NL are extracted from healthy individuals with normal methylation, hence only U primers give rise to positive amplification. The in vitro methylation process converts the unmethylated DNA to methylated DNA, resulting in positive amplification with M primers. The U bands in IVD are due to incomplete conversion, which is frequently observed. The on-chip MSP assay was analyzed by fluorescence imaging (FIG. 5b-5e). Dark spots in the images indicated DNA amplification. Positive amplification of NL was only observed with U primers (FIG. 5b). In contrast, IVD was successfully amplified by M primers. Since the in vitro methylation conversion was partial, both U and M reactions gave positive results (FIG. 5c). With the reaction volume being substantially reduced, the results obtained using our micro MSP chip agreed with the results from control reactions (FIG. 5a).

The majority of the micro PCR chips were closed chamber systems, which required filling up the entire reaction chamber with sample mixture. Therefore, the reaction volume was defined by the chamber size and fixed once the device was fabricated. In contrast, the open chamber device was more flexible and allowed for a wide range of reaction volumes. The reaction chamber did not define the size of the droplet but merely served as containment. To demonstrate that the reaction volume could be further reduced using the same device, a total of 10 μl, MSP mixture was used in this case which resulted in submicroliter reaction volume. The MSP results from NL and IVD again agreed with control reactions (FIG. 5a) although smaller spots were observed due to the reduction in reaction volumes (FIGS. 5d and 5e).

### Advantages

The open access facilitates primer deposition. With pre-deposited primers, the sample preparation procedure is greatly simplified and the throughput is significantly improved. Currently, the design allows for preparing 108 reactions with 9 sample injections. It is possible to further reduce the number of injections by integrating more reactions chambers into a single snowflake-like functional unit. The open chamber design also provides easy access to surface modification and post PCR sample retrieval for downstream analysis. Bubble formation and expansion were the prominent problems that complicate many closed chamber PCR devices. Bubbles can easily expand and push the sample out of the chamber during heating. Therefore, bubble containment modules are often incorporated for successful PCR reactions. Open chamber design, on the other hand, circumvents this problem, not by eliminating bubbles but by tolerating bubble formation. The tolerance to bubbles eases the sample loading process. Since no additional equipment is required for operating bubble containment module and microfluidics, our micro MSP chip is compatible with conventional bench-top thermal cyclers with minor modification and can be easily handled by individuals who are not experienced in microfluidics. The simple design also provides easy portability. Along with chemicals and reagents, the micro MSP chips can be provided in the form of diagnostic kits for clinical laboratory applications. Although the reaction chambers are open, the MSP reactions are performed in a sealed format. The companion fluid compartmentalizes the samples by encapsulating them into droplets. These sample droplets are completely covered by the companion fluid and isolated from the environment, therefore the chance of contamination is minimized.

This platform provides a simple and relatively high throughput method to prepare droplets for biochemical reactions.

### CONCLUSION

The results presented above all indicate that our constructed platform confers unprecedented ease in sample handling due to the novel sample loading scheme, which offers a simple way to prepare multiple droplets for biochemical reactions. By pre-depositing primers and using immiscible oil as companion fluid, it allows multiple reactions with just single sample injection. MSP assays are performed in sealed micro droplets that isolate the reactions from the environment and prevent contamination. The open chamber design facilitates easy surface modification, primer deposition and sample retrieval. It also bypasses the need for a bubble containment mechanism. In addition, the proposed platform allows reliable MSP reactions in a sub-microliter volume as opposed to the typical volume of 50 μL for MSP. The downscaling of MSP assays by nearly two orders of magnitude greatly reduces the consumption of reagent and valuable samples. Furthermore, this platform allows multiple DNA methylation analyses performed in an array-based manner. We expect the device to be implemented as a versatile miniaturized PCR platform applicable to many other PCR-based genomic analysis assays. The aforementioned advantages may make the proposed platform widely adopted by both clinical and research laboratories.

References cited herein are listed below for convenience and are hereby incorporated by reference in their entirety.
1. C. R. Newton, A. Graham, L. E. Heptinstall, S. J. Powell, C. Summers, N. Kalsheker, J. C. Smith and A. F. Markham, Nucleic Acids Res., 1989, 17, 2503-2516.
2. J. E. Rice, J. A. Sanchez, K. E. Pierce, A. H. Reis, A. Osborne and L. J. Wangh, Nature Protocols, 2007, 2, 2429-2438.
3. C. A. Heid, J. Stevens, K. J. Livak and P. M. Williams, Genome Research, 1996, 6, 986-994.
4. F. Diehl, M. Li, Y. P. He, K. W. Kinzler, B. Vogelstein and D. Dressman, Nat. Methods., 2006, 3, 551-559.
5. M. Margulies, M. Egholm, W. E. Altman, S. Attiya, J. S. Bader, L. A. Bemben, J. Berka, M. S. Braverman, Y. J. Chen, Z. T. Chen, S. B. Dewell, L. Du, J. M. Fierro, X. V. Gomes, B. C. Godwin, W. He, S. Helgesen, C. H. Ho, G. P. Irzyk, S. C. Jando, M. L. I. Alenquer, T. P. Jarvie, K. B. Jirage, J. B. Kim, J. R. Knight, J. R. Lanza, J. H. Leamon, S. M. Lefkowitz, M. Lei, J. Li, K. L. Lohman, H. Lu, V. B. Makhijani, K. E. McDade, M. P. McKenna, E. W. Myers, E. Nickerson, J. R. Nobile, R. Plant, B. P. Puc, M. T. Ronan, G. T. Roth, G. J. Sarkis, J. F. Simons, J. W. Simpson, M. Srinivasan, K. R. Tartaro, A. Tomasz, K. A. Vogt, G. A. Vollmer, S. H. Wang, Y. Wang, M. P. Weiner, P. G. Yu, R. F. Begley and J. M. Rothberg, Nature, 2005, 437, 376-380.
6. J. G. Herman, J. R. Graff, S. Myohanen, B. D. Nelkin and S. B. Baylin, Proc. Natl. Acad. Sci. U.S.A, 1996, 93, 9821-9826.
7. H. Craighead, Nature, 2006, 442, 387-393.
8. A. J. deMello, Nature, 2006, 442, 394-402.
9. J. El-Ali, P. K. Sorger and K. F. Jensen, Nature, 2006, 442, 403-411.
10. D. Janasek, J. Franzke and A. Manz, Nature, 2006, 442, 374-380.
11. G. M. Whitesides, Nature, 2006, 442, 368-373.
12. P. Yager, T. Edwards, E. Fu, K. Helton, K. Nelson, M. R. Tam and B. H. Weigl, Nature, 2006, 442, 412-418.
13. P. A. Auroux, Y. Koc, A. deMello, A. Manz and P. J. R. Day, Lab Chip, 2004, 4, 534-546.
14. L. J. Kricka and P. Wilding, Analytical and Bioanalytical Chemistry, 2003, 377, 820-825.
15. C. S. Zhang and D. Xing, Nucleic Acids Res., 2007, 35, 4223-4237.
16. M. A. Burns, B. N. Johnson, S, N. Brahmasandra, K. Handique, J. R. Webster, M. Krishnan, T. S. Sammarco, P. M. Man, D. Jones, D. Heldsinger, C. H. Mastrangelo and D. T. Burke, Science, 1998, 282, 484-487.
17. M. Krishnan, D. T. Burke and M. A. Burns, Analytical Chemistry, 2004, 76, 6588-6593.
18. D. S. Lee, S. H. Park, H. S. Yang, K. H. Chung, T. H. Yoon, S. J. Kim, K. Kim and Y. T. Kim, Lab on a Chip, 2004, 4, 401-407.
19. J. Liu, C. Hansen and S. R. Quake, Analytical Chemistry, 2003, 75, 4718-4723.
20. M. A. Northrup, B. Benett, D. Hadley, P. Landre, S. Lehew, J. Richards and P. Stratton, Analytical Chemistry, 1998, 70, 918-922.
21. A. T. Woolley, D. Hadley, P. Landre, A. J. deMello, R. A. Mathies and M. A. Northrup, Analytical Chemistry, 1996, 68, 4081-4086.
22. P. Neuzil, C. Y. Zhang, J. Pipper, S. Oh and L. Zhuo, Nucleic Acids Research, 2006, 34.
23. J. Pipper, M. Inoue, L. F. P. Ng, P. Neuzil, Y. Zhang and L. Novak, Nat. Med., 2007, 13, 1259-1263.
24. J. Pipper, Y. Zhang, P. Neuzil and T. M. Hsieh, Angewandte Chemie-International Edition, 2008, 47, 3900-3904.
25. H. B. Liu, H. Q. Gong, N. Ramalingam, Y. Jiang, C. C. Dai and K. M. Hui, J. Micromech. Microeng., 2007, 17, 2055-2064.
26. T. Nakayama, Y. Kurosawa, S. Furui, K. Kerman, M. Kobayashi, S. R. Rao, Y. Yonezawa, K. Nakano, A. Hino, S. Yamamura, Y. Takamura and E. Tamiya, Anal. Chem., 2006, 386, 1327-1333.
27. A. Bird, Genes Dev., 2002, 16, 6-21.
28. J. G. Herman and S. B. Baylin, New England Journal of Medicine, 2003, 349, 2042-2054.
29. P. A. Jones and S. B. Baylin, Nat. Rev. Genet., 2002, 3, 415-428.
30. P. A. Jones and P. W. Laird, Nature Genetics, 1999, 21, 163-167.
31. V. E. A. Russo, R. A. Martienssen and A. D. Riggs, Epigenetic mechanisms of gene regulation, Cold Spring Harbor Laboratory Press, Plainview, N.Y., 1996.
32. R. Holliday and G. W. Grigg, Mutation Research, 1993, 285, 61-67.
33. S. B. Baylin and J. G. Herman, Trends in Genetics, 2000, 16, 168-174.
34. M. V. Brock, C. M. Hooker, E. Ota-Machida, Y. Han, M. Z. Guo, S. Ames, S. Glockner, S. Piantadosi, E. Gabrielson, G. Pridham, K. Pelosky, S. A. Belinsky, S.C. Yang, S. B. Baylin and J. G. Herman, New England Journal of Medicine, 2008, 358, 1118-1128.
35. M. V. Brock, J. G. Herman and S. B. Baylin, New England Journal of Medicine, 2008, 358, 2514-2514.
36. M. Esteller, J. Garcia-Foncillas, E. Andion, S, N. Goodman, O. F. Hidalgo, V. Vanaclocha, S. B. Baylin and J. G. Herman, New England Journal of Medicine, 2000, 343, 1350-1354.
37. Y. Zhang, V. Bailey, C. M. Puleo, C. Chen and T. H. Wang, microTAS 2008 conference proceeding, 2008.
38. Y. Z. Vasudev J. Bailey, Hariharan Easwaran, Elizabeth Griffiths, James G. Herman, Stephen B. Baylin, Hetty E. Carraway, Tza-Huei Wang, in AACR conference “Molecular Diagnostics in Cancer Therapeutic Development”, Philadelphia, 2008.
39. P. Cooley, D. Wallace and B. Antohe, Journal of the Association for Laboratory Automation, 2002, 7, 33-39.
40. E. O. Machida, M. V. Brock, C. M. Hooker, J. Nakayama, A. Ishida, J. Amano, M. A. Picchi, S. A. Belinsky, J. G. Herman, S. Taniguchi and S. B. Baylin, Cancer Res., 2006, 66, 6210-6218.
41. O. G. a. J. G. Herman, Multiple Myeloma, Humana Press, 2005.
42. Z. Q. Niu, W. Y. Chen, S. Y. Shao, X. Y. Jia and W. P. Zhang, Journal of Micromechanics and Microengineering, 2006, 16, 425-433.
43. N. M. Toriello, C. N. Liu and R. A. Mathies, Analytical Chemistry, 2006, 78, 7997-8003.
44. J. F. Chen, M. Wabuyele, H. W. Chen, D. Patterson, M. Hupert, H. Shadpour, D. Nikitopoulos and S. A. Soper, Analytical Chemistry, 2005, 77, 658-666.
45. Z. Y. Chen, J. Wang, S. Z. Qian and H. H. Bau, Lab on a Chip, 2005, 5, 1277-1285.
46. Y. S. Shin, K. Cho, S. H. Lim, S. Chung, S. J. Park, C. Chung, D.C. Han and J. K. Chang, Journal of Micromechanics and Microengineering, 2003, 13, 768-774.
47. N.C. Cady, S. Stelick, M. V. Kunnavakkam and C. A. Batt, Sensors and Actuators B-Chemical, 2005, 107, 332-341.
48. E. T. Lagally, I. Medintz and R. A. Mathies, Analytical Chemistry, 2001, 73, 565-570.
49. T.-M. Hsieh, Y. Zhang, J. Pipper and P. Neuzil, microTAS 2006 conference proceeding, 2006.
50. K. D. Dorfman, M. Chabert, J. H. Codarbox, G. Rousseau, P. de Cremoux and J. L. Viovy, Analytical Chemistry, 2005, 77, 3700-3704.
51. U. Lehmann, C. Vandevyver, V. K. Parashar and M. A. M. Gijs, Angewandte Chemie-International Edition, 2006, 45, 3062-3067.
52. J. Wen et. al, Anal. Chem. 2008, 80, 6472-6479
53. E. T. Lagally et. al., Anal. Chem. 2004, 76, 3162-3170
54. L. A. Legendre et al., Anal. Chem. 2006, 78, 1444-1451
55. Y. Zhang et. al., Lab Chip, 2009, 9, 1059-1064

We claim:

1. A device comprising a sampling chamber having an access point and connected to at least one or more channels extending radially outward, each of said channels terminating at an open reaction chamber containing a pre-deposited reaction material.

2. The device of claim 1, wherein the sampling chamber is connected to about six channels.

3. The device of claim 1, wherein each channel branches off into two or more secondary channels, each of said secondary channels connecting to an open reaction chamber.

4. The device of claim 1, wherein the device comprises about 12 open reaction chambers.

5. The device of claim 1, wherein the reaction material is a pre-deposited solidified material.

6. The device of claim 1, wherein the material is a PCR primer.

7. The device of claim 1, wherein the material is an unmethylated primer.

8. The device of claim 1, wherein the material is a primer specific for a bisulfite modified unmethylated sequence.

9. The device of claim 1, wherein the material is a methylated primer.

10. The device of claim 1, wherein the material is a primer specific for a bisulfite modified methylated sequence.

11. The device of claim 1, wherein the device comprises two or more open reaction chambers, said chambers containing similar material.

12. The device of claim 1, wherein the device comprises two or more open reaction chambers, each of said chambers containing two or more different material.

13. A method of making a device of claim 1, comprising:
a. depositing a reaction mixture comprising a solvent and a reaction material onto the bottom surface of the designated open reaction chamber, near the outlet of the channel connected to said open reaction chamber, and
b. allowing evaporation of solvent and the adsorption of the reaction material to form a pre-deposited solidified material.

14. The method of claim 13, wherein, the device is stored at 4 degrees Celsius until use.

15. A method of analyzing a biological sample using the device of claim 1, comprising:
a. introducing a biological sample through the access point and into the channels,
b. introducing a immiscible companion fluid to force the biological sample that are inside the channels into the open reaction chambers,
c. allowing the re-suspension of the pre-deposited material,
d. allowing for a biochemical mixture of the biological sample with the re-suspended pre-deposited material,
e. allowing for a biochemical reaction to occur between the biological sample and the re-suspended pre-deposited material, and
f. performing a laboratory diagnostic test of the biochemical mixture.

16. The method of claim 15, further comprising introducing a second amount of immiscible companion fluid to prevent possible backflow and cross contamination of said biochemical mixture.

17. The method of claim 15, wherein after step (b), the biological sample forms droplets in the immiscible companion fluid.

18. The method of claim 15, wherein the immiscible companion fluid encapsulates and insulates the biochemical reaction mixture of step (d) to prevent direct evaporation.

19. The method of claim 15, wherein the biological sample is purified DNA.

20. The method of claim 15, wherein the immiscible companion fluid is immiscible mineral oil.

21. The method of claim 15, wherein the biological sample is introduced using an injection needle.

22. The method of claim 15, wherein the laboratory diagnostic test is DNA methylation detection.

23. A kit comprising:
a. the device of claim 1,
b. chemical reagents for containing a biological sample, and
c. an immiscible companion fluid.

24. The method of claim 23, further comprising one or more instruments for introducing the biological sample and chemical reagent mixture and the immiscible companion fluid.


SEQUENCE LISTING (8 sequences)

SEQ ID NO: 1 - DNA, 30 bp, Artificial Sequence
  Description of Artificial Sequence Synthetic primer
ggttggttttttattttgttagagtgaggt

SEQ ID NO: 2 - DNA, 29 bp, Artificial Sequence
  Description of Artificial Sequence Synthetic primer
aaccactctaaccacaaaatacaaacaca

SEQ ID NO: 3 - DNA, 26 bp, Artificial Sequence
  Description of Artificial Sequence Synthetic primer
ggttttttattttgttagagcgaggc

SEQ ID NO: 4 - DNA, 21 bp, Artificial Sequence
  Description of Artificial Sequence Synthetic primer
taaccgcaaaatacgaacgcg

SEQ ID NO: 5 - DNA, 27 bp, Artificial Sequence
  Description of Artificial Sequence Synthetic primer
gaaggtggggagtttaggttttgtttt

SEQ ID NO: 6 - DNA, 28 bp, Artificial Sequence
  Description of Artificial Sequence Synthetic primer
aaattctccaacacatccaaaataacat

SEQ ID NO: 7 - DNA, 23 bp, Artificial Sequence
  Description of Artificial Sequence Synthetic primer
gcggggagtttaggtttcgtttc

SEQ ID NO: 8 - DNA, 23 bp, Artificial Sequence
  Description of Artificial Sequence Synthetic primer
ccaacgcatccaaaataacgtcg